(19) United States
(12) Patent Application Publication
(10) Pub. No.: US 2025/0185931 A1
(43) Pub. Date: Jun. 12, 2025

(54) HOUSING, MANUFACTURING METHOD FOR HOUSING, AND ELECTRONIC DEVICE

(71) Applicant: GUANGDONG OPPO MOBILE TELECOMMUNICATIONS CORP., LTD., Dongguan (CN)

(72) Inventors: Yingchao WU, Dongguan (CN); Jianwen Lin, Dongguan (CN)

(73) Assignee: GUANGDONG OPPO MOBILE TELECOMMUNICATIONS CORP., LTD., Dongguan (CN)

(21) Appl. No.: 18/979,122

(22) Filed: Dec. 12, 2024

Related U.S. Application Data
(63) Continuation of application No. PCT/CN2022/138785, filed on Dec. 13, 2022.

(30) Foreign Application Priority Data
Jun. 14, 2022 (CN) 202210672787.0

(51) Int. Cl. A61B 5/024

(52) U.S. Cl. CPC A61B 5/02433; A61B 5/02438

(57) ABSTRACT

A housing, a manufacturing method for the housing, and an electronic device. The housing includes a housing body, a first light-transmitting part, a second light-transmitting part spaced apart from the first light-transmitting part, and a light-shielding baffle arranged between the first light-transmitting part and the second light-transmitting part. At least one of the following is satisfied: at least a portion of an outer peripheral edge of the first light-transmitting part is directly connected to the housing body; and at least a portion of the outer peripheral edge of the second light-transmitting part is directly connected to the housing body.

The present application claims priority of Chinese Patent Application No. 202210672787.0, in the title of “HOUSING, MANUFACTURING METHOD FOR HOUSING, AND ELECTRONIC DEVICE”, filed on Jun. 14, 2022, the entire contents of which are hereby incorporated by reference in their entirety.

### TECHNICAL FIELD

The present disclosure relates to the technical field of electronics, in particular to a housing, a method for manufacturing a housing, and an electronic device.

### BACKGROUND

With people's growing health awareness, more and more smart wearable devices (such as smart watches, smart bracelets, etc.) are arranged to integrate optical components, so as to measure physiological data such as heart rate and blood oxygen concentration based on the Photoplethysmography (PPG) principle.

A smart wearable device usually includes a housing, a light emitter, and a light detector. The housing is arranged with a first light-transmitting part and a second light-transmitting part. The light signal emitted by the light emitter can pass through the first light-transmitting part to reach the human skin, and the light signal reflected from the human skin can pass through the second light-transmitting part to reach the light detector. The physiological data of the human body can be obtained after processing the light signal detected by the light detector. However, when the light emitted by the light emitter, that is supposed to be transmitted into the first light-transmitting part enters the second light-transmitting part and is received by the light detector, that is, when the light signal emitted by the light emitter is directly received by the light detector without being reflected by the skin, an error will occur in the light signal detected by the light detector, which in turn will reduce the accuracy of the test results.

### SUMMARY OF THE DISCLOSURE

The embodiments of the present disclosure provide a housing, a method for manufacturing a housing, and an electronic device. The housing can prevent crosstalk between the light in the first light-transmitting part and the light in the second light-transmitting part. When the housing is applied in an electronic device, the accuracy of the detection results of the electronic device can be improved.

In a first aspect, the present disclosure provides a housing, including:
a housing body;
a first light-transmitting part;
a second light-transmitting part, spaced apart from the first light-transmitting part; and
a light-shielding baffle, arranged between the first light-transmitting part and the second light-transmitting part;
wherein at least one of the following is satisfied:
at least a portion of an outer peripheral edge of the first light-transmitting part is directly connected to the housing body; and
at least a portion of the outer peripheral edge of the second light-transmitting part is directly connected to the housing body;
wherein at least one of the following is satisfied:
an absolute value of a difference between a thermal expansion coefficient of a material of the first light-transmitting part and a thermal expansion coefficient of a material of the housing body is a first difference, and a ratio of the first difference to the thermal expansion coefficient of the material of the housing body is less than or equal to 20%; and
an absolute value of a difference between a thermal expansion coefficient of a material of the second light-transmitting part and a thermal expansion coefficient of a material of the housing body is a second difference, and a ratio of the second difference to the thermal expansion coefficient of the material of the housing body is less than or equal to 20%.

In a second aspect, the present disclosure provides a method for manufacturing the housing as above, including:
arranging the material of the housing body, the material the first light-transmitting part, the material the second light-transmitting part, and the material the light-shielding baffle in accordance with positions of the housing body, the first light-transmitting part, the second light-transmitting part, and the light-shielding baffle; and
extruding the material of the housing body, the material of the first light-transmitting part, the material of the second light-transmitting part, and the material of the light-shielding baffle to connect the material of the housing body, the material of the first light-transmitting part, the material of the second light-transmitting part, and the material of the light-shielding baffle, for obtaining the housing.

In a third aspect, the present disclosure provides an electronic device, including:
the housing as above;
a light emitter, arranged on a side of the housing; wherein light emitted by the light emitter is configured to enter the first light-transmitting part and be transmitted to another side of the housing away from the light emitter via the first light-transmitting part; and
a light detector; wherein the light detector and the light emitter are arranged on a same side of the housing, and the light incident from the side of the housing away from the light detector is configured to enter the second light-transmitting part, be transmitted through the second light-transmitting part, and then enter the light detector.

### BRIEF DESCRIPTION OF THE DRAWINGS

In order to more clearly illustrate the technical solution in the embodiments of the present disclosure, the following is a brief description of the drawings needed to be used in the description of the embodiments. Obviously, the drawings described below are only some embodiments of the present disclosure. For those skilled in the art, without the expenditure of creative labor, other drawings can be obtained based on these drawings.

In order to more completely understand the present disclosure and its beneficial effects, the following description will be combined with the accompanying drawings, in which the same accompanying figure number indicates the same part in the following description.

FIG. 1 is a first structural schematic view of a housing according to some embodiments of the present disclosure.

FIG. 2 is a second structural schematic view of a housing according to some embodiments of the present disclosure.

FIG. 3 is a third structural schematic view of a housing according to some embodiments of the present disclosure.

FIG. 4 is a fourth structural schematic view of a housing according to some embodiments of the present disclosure.

FIG. 5 is a fifth structural schematic view of a housing according to some embodiments of the present disclosure.

FIG. 6 is a sixth structural schematic view of a housing according to some embodiments of the present disclosure.

FIG. 7 is a seventh structural schematic view of a housing according to some embodiments of the present disclosure.

FIG. 8 is an eighth structural schematic view of a housing according to some embodiments of the present disclosure.

FIG. 9 is a ninth structural schematic view of a housing according to some embodiments of the present disclosure.

FIG. 10 is a tenth structural schematic view of a housing according to some embodiments of the present disclosure.

FIG. 11 is an eleventh structural schematic view of a housing according to some embodiments of the present disclosure.

FIG. 12 is a flowchart of a method for manufacturing a housing according to some embodiments of the present disclosure.

FIG. 13 is a schematic diagram which provides an arrangement of materials of a housing body, a first light-transmitting part, a second light-transmitting part, and a light-shielding baffle according to positions of the housing body, the first light-transmitting part, the second light-transmitting part, and the light-shielding baffle in FIG. 1, according to some embodiments of the present disclosure.

FIG. 14 is a schematic diagram which provides an arrangement of materials of a housing body, a first light-transmitting part, a second light-transmitting part, and a light-shielding baffle according to positions of the housing body, the first light-transmitting part, the second light-transmitting part, and the light-shielding baffle in FIG. 3, according to some embodiments of the present disclosure.

FIG. 15 is a schematic diagram which provides an arrangement of materials of a housing body, a first light-transmitting part, a second light-transmitting part, and a light-shielding baffle according to positions of the housing body, the first light-transmitting part, the second light-transmitting part, and the light-shielding baffle in FIG. 5, according to some embodiments of the present disclosure.

FIG. 16 is a structural schematic view of a composite rod body according to some embodiments of the present disclosure.

FIG. 17 is a partially structural schematic view of an electronic device according to some embodiments of the present disclosure.

### DETAILED DESCRIPTION

The following description of the technical solutions in the embodiments of the present disclosure will be provided in a clear and complete manner with reference to the accompanying drawings in the embodiments of the present disclosure. Obviously, the embodiments described are only some embodiments of the present disclosure, but not all of the embodiments. Based on the embodiments in the present disclosure, all other embodiments obtained by those skilled in the art without creative effort fall within the scope of the present disclosure.

References in this document to “embodiments” or “implementations” mean that particular features, structures, or characteristics described in conjunction with an embodiment or implementation may be included in at least one embodiment of the present disclosure. The occurrence of the phrase in various places in the specification does not necessarily refer to the same embodiment, nor is it mutually exclusive with other embodiments, independent or alternative embodiments. It is expressly and implicitly understood by those skilled in the art that the embodiments described herein may be combined with other embodiments.

Referring to FIG. 1, FIG. 1 is a first structural schematic view of a housing according to some embodiments of the present disclosure. The present embodiments provide a housing 110, including a housing body 10, a first light-transmitting part 20, a second light-transmitting part 30, and a light-shielding baffle 40; where the second light-transmitting part 30 is arranged spaced apart from the first light-transmitting part 20, and the light-shielding baffle 40 is arranged between the first light-transmitting part 20 and the second light-transmitting part 30.

In particular, at least a portion of an outer peripheral edge of the first light-transmitting part 20 is directly connected to the housing body 10, and at least a portion of the outer peripheral edge of the second light-transmitting part 30 is directly connected to the housing body 10.

The ratio of the absolute value of the difference between the thermal expansion coefficients of the material of the first light-transmitting part 20 and the material of the housing body 10 to the thermal expansion coefficient of the material of the housing body 10 is less than or equal to 20% (e.g., the ratio is 20%, 17%, 15%, 12%, 10%, 8%, 5%, 3%, 1%, 0.5%, 0, etc.).

The ratio of the absolute value of the difference between the thermal expansion coefficients of the material of the second light-transmitting part 30 and the material of the housing body 10 to the thermal expansion coefficient of the material of the housing body 10 is less than or equal to 20% (e.g., the ratio is 20%, 17%, 15%, 12%, 10%, 8%, 5%, 3%, 1%, 0.5%, 0, etc.).

It should be noted that in the embodiments of the present disclosure, the direct connection of the first light-transmitting part 20, the second light-transmitting part 30, and the housing body 10 refers to the first light-transmitting part 20, the second light-transmitting part 30, and the housing body 10 being a one-piece structure, where the junction of the first light-transmitting part 20, the second light-transmitting part 30, and the housing body 10 is formed with a continuous material interface, rather than a combined structure (e.g., inlay, adhesion, etc.).

For example, in the housing 110 provided in the embodiments of the present disclosure, the housing body 10, the first light-transmitting part 20, the second light-transmitting part 30, and the light-shielding baffle 40 are prepared by means of one-piece molding.

It can be understood that when the ratio of the absolute value of the difference between the thermal expansion coefficients of the material of the first light-transmitting part 20 and the material of the housing body 10 to the thermal expansion coefficient of the material of the housing body 10 is less than or equal to 20% and the ratio of the absolute value of the difference between the thermal expansion coefficients of the material of the second light-transmitting part 30 and the material of the housing body 10 to the thermal expansion coefficient of the material of the housing body 10 is less than or equal to 20%, the difference between the thermal expansion coefficients of the materials of the first light-transmitting part 20 and the second light-transmitting part 30 and the thermal expansion coefficient of the material of the housing body 10 is very small. Therefore, when the material of the housing body 10, the material of the first light-transmitting part 20, and the material of the second light-transmitting part 30 are softened by heating, the expansion rates of the material of the housing body 10, the material of the first light-transmitting part 20, and the material of the second light-transmitting part 30 are similar. After the material of the housing body 10, the material of the first light-transmitting part 20, and the material of the second light-transmitting part 30 are compressed together and cooled, the shrinkage rates of the material of the housing body 10, the material of the first light-transmitting part 20, and the material of the second light-transmitting part 30 are similar. In this way, the parts where the housing body 10 is connected to the first light-transmitting part 20 and the second light-transmitting part 30 are not prone to cracking, thereby improving the mechanical strength of the housing 110.

It should be noted that the housing 110 in the embodiments of the present disclosure can pass the steel ball impact test (where a steel ball weighing 110 g is dropped freely from a height of 5 cm above the surface of the housing 110 to impact the first light-transmitting part 20 and the second light-transmitting part 30 on the housing 110). The results of the steel ball impact test show that after the steel ball impacts the housing 110, no cracking appears on the surface of the housing 110. In other words, the housing 110 provided by the present embodiments has high mechanical strength and good impact resistance.

In addition to testing the housing 110 of the present disclosure, the Inventor further tests a housing of the prior art in which the periphery of the first light-transmitting part and the periphery of the second light-transmitting part are surrounded by a light-shielding material. The results of the steel ball impact test show that, after the steel ball impact, cracking appears between the first light-transmitting part of the housing and the surrounding light-shielding material, and cracking also appears between the second light-transmitting part and the surrounding light-shielding material. In other words, this housing cannot pass the steel ball impact test and has low mechanical strength and poor impact resistance.

Schematically, the light transmittance of the first light-transmitting part 20 and the second light-transmitting part 30 for light with a wavelength of 400 nm to 1200 nm is greater than or equal to 30%, for example 30%, 40%, 50%, 60%, 70%, 80%, 90% or 100%; for example, the light-shielding baffle 40 has a light transmittance of less than or equal to 20% for light with a wavelength of 400 nm to 1200 nm, for example 20%, 18%, 15%, 12%, 10%, 8%, 5%, 3%, 1% or 0%.

Schematically, the material 71 of the housing body 10, the material 72 of the first light-transmitting part 20, the material 73 of the second light-transmitting part 30, and the material 74 of the light-shielding baffle 40 can each be made of glass or resin. In particular, the material 71 of the housing body 10, the material 72 of the first light-transmitting part 20, and the material 73 of the second light-transmitting part 30 may each be colorless and transparent, and the material 74 of the light-shielding baffle 40 may be black.

Schematically, the material 71 of the housing body 10, the material 72 of the first light-transmitting part 20, and the material 73 of the second light-transmitting part 30 may be the same, for example, the material 71 of the housing body 10, the material 72 of the first light-transmitting part 20, and the material 73 of the second light-transmitting part 30 may all be made of glass or all be made of resin.

It can be understood that when different materials (with different thermal expansion coefficients) are combined by heating and softening and extruding to form a housing, the different materials are prone to separate from each other after cooling, thus causing the housing to crack, due to the different thermal expansion coefficients of the different materials. In the embodiments of the present disclosure, since the first light-transmitting part 20 and the second light-transmitting part 30 are directly connected to the housing body 10, when the material 71 of the housing body 10, the material 72 of the first light-transmitting part 20, and the material 73 of the second light-transmitting part 30 are the same, and the thermal expansion coefficients of the same materials are the same, therefore after the material 71 of the housing body 10, the material 73 of the second light-transmitting part 30, and the material 72 of the first light-transmitting part 20 are softened by heating and pressed together, cracking is not likely to occur, thereby enhancing the mechanical strength of the housing 110.

Referring to FIG. 1, multiple second light-transmitting parts 30 may be arranged, and the multiple second light-transmitting parts 30 are arranged around the first light-transmitting part 20. In the embodiments of the present disclosure, multiple refers to two or more, such as three, four, five, six, seven, eight, nine, ten, etc.

Schematically, the first light-transmitting part 20 and the second light-transmitting part 30 may each be a figure such as a regular hexagon, regular pentagon, square, rectangle, equilateral triangle, circle, ellipse, sector, or irregular shape.

Referring to FIG. 1, the light-shielding baffle 40 may include a first baffle 41 and a second baffle 42; the first baffle 41 is arranged around the first light-transmitting part 20, the first baffle 41 defines one or more first notches 411, and the first light-transmitting part 20 is directly connected to the housing body 10 at the one or more first notches 411; the second baffle 42 is arranged between each two adjacent second light-transmitting parts 30.

It can be understood that by providing the second baffle 42 between adjacent second light-transmitting parts 30, light crosstalk between adjacent second light-transmitting parts 30 may be avoided. Since the first light-transmitting part 20 is directly connected to the housing body 10 at the first notches 411, when the first light-transmitting part 20 and the housing body 10 are extruded using the same material (i.e., the thermal expansion coefficients of the materials are the same), the mechanical strength of the housing 110 can be significantly improved, such that the housing 110 is less likely to crack.

Referring to FIG. 1, each second baffle 42 may be arranged facing a corresponding first notch 411, and the second baffle 42 is not connected to the first baffle 41. It can be seen that in this case, a portion of the housing body 10 is filled between the second baffle 42 and the first baffle 41, such that the housing body 10 can be connected to the first light-transmitting part 20 at the first notch 411.

Referring to FIG. 1, all regions of the outer peripheral edge of the second light-transmitting part 30 are directly connected to the housing body 10. When the second light-transmitting part 30 and the housing body 10 are extruded from the same material (i.e., the thermal expansion coefficients of the materials are the same), the mechanical strength of the housing 110 can be further enhanced, such that the housing 110 is less prone to cracking.

Schematically, the number of the second baffles 42 may be at least one, i.e., one or more, depending on the number of second light-transmitting parts 30.

Referring to FIG. 1, the number of the second baffles 42 may be multiple, and the multiple second baffles 42 are distributed radially around the first light-transmitting part 20. For example, there is an interval between an end of the second baffle 42 away from the first light-transmitting part 20 and the edge of the housing body 10, i.e., the second baffle 42 does not extend to the edge of the housing body 10, thereby preventing the housing body 10 from being split into multiple parts and ensuring that the housing body 10 is a continuous one-piece structure.

Referring to FIG. 1, the first baffle 41 may define multiple first notches 411, and the first baffle 41 includes multiple first baffle units 412 arranged at intervals, with a first notch 411 defined between each adjacent two first baffle units 412. Exemplarily, the multiple first baffle units 412 may be disposed on the edges of figures such as regular hexagons, regular pentagons, squares, rectangles, equilateral triangles, circles, ellipses, sectors, irregular shapes, etc.

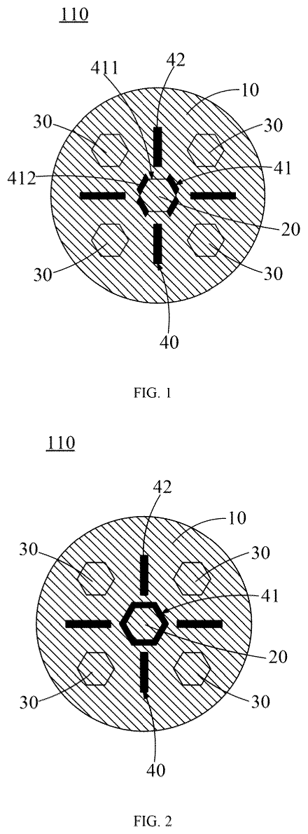

Referring to FIG. 2, FIG. 2 is a second structural schematic view of a housing according to some embodiments of the present disclosure. FIG. 2 differs from FIG. 1 in that in FIG. 1, the first baffle 41 defines a first notch 411, and the first light-transmitting part 20 is directly connected to the housing body 10 at the first notch 411, while in FIG. 2, the first baffle 41 is in a closed state. That is, the first notch 411 is not provided on the first baffle 41 in FIG. 2, and the first light-transmitting part 20 is not connected to the housing body 10. However, in FIG. 2, all regions of the outer peripheral edge of the second light-transmitting part 30 are directly connected to the housing body 10, and the ratio of the absolute value of the difference between the thermal expansion coefficients of the material of the second light-transmitting part 30 and the material of the housing body 10 to the thermal expansion coefficient of the material of the housing body 10 is less than or equal to 20% (e.g., the ratio is 20%, 17%, 15%, 12%, 10%, 8%, 5%, 3%, 1%, 0.5%, 0, etc.). It can be understood that by setting the second light-transmitting part 30 to be directly connected to the housing body 10, since the difference between the thermal expansion coefficients of the materials of the second light-transmitting part 30 and the housing body 10 is very small, the part where the housing body 10 and the second light-transmitting part 30 are connected is not prone to cracking, thereby enhancing the mechanical strength of the housing 110.

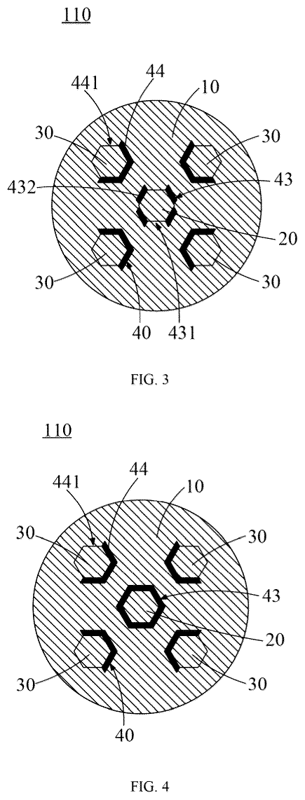

Referring to FIG. 3, FIG. 3 is a third structural schematic view of a housing according to some embodiments of the present disclosure. The light-shielding baffle 40 may include a third baffle 43 and a fourth baffle 44. The third baffle 43 is arranged around the first light-transmitting part 20, and the third baffle 43 defines one or more third notches 431, where the first light-transmitting part 20 is directly connected to the housing body 10 at the one or more third notches 431; the fourth baffle 44 is arranged around each second light-transmitting part 30, and the fourth baffle 44 defines one or more fourth notches 441, where the second light-transmitting part 30 is directly connected to the housing body 10 at the one or more fourth notches 441.

It can be understood that when the first light-transmitting part 20 is directly connected to the housing body 10 at the third notch 431, the second light-transmitting part 30 is directly connected to the housing body 10 at the fourth notch 441, and the first light-transmitting part 20, the second light-transmitting part 30 and the housing body 10 are extruded from the same material (i.e., the thermal expansion coefficients of the materials are the same), the mechanical strength of the housing 110 can be significantly improved, and the housing 110 is less prone to cracking.

Referring to FIG. 3, the third baffle 43 may define multiple third notches 431, and the third baffle 43 includes multiple third baffle units 432 arranged at intervals, with a third notch 431 defined between each adjacent two third baffle units 432. Exemplarily, the multiple third baffle units 432 may be disposed on the edges of figures such as regular hexagons, regular pentagons, squares, rectangles, equilateral triangles, circles, ellipses, sectors, irregular shapes, etc.

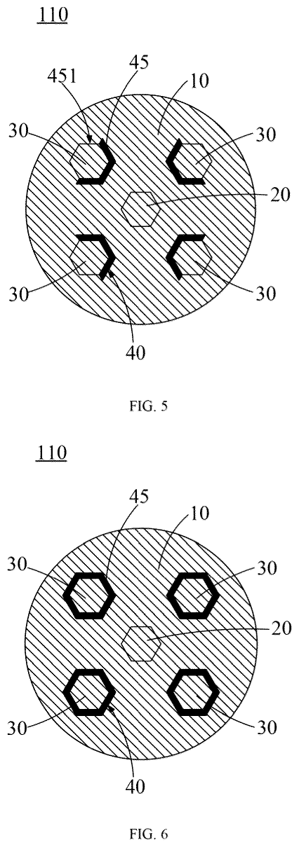

Referring to FIG. 4, FIG. 4 is a fourth structural schematic view of a housing according to some embodiments of the present disclosure. FIG. 4 differs from FIG. 3 in that in FIG. 3, a third notch 431 is defined on the third baffle 43, and the first light-transmitting part 20 is directly connected to the housing body 10 at the third notch 431, while in FIG. 4, the third baffle 43 is in a closed state. That is, the third notch 431 is not provided on the third baffle 43, and the first light-transmitting part 20 is not connected to the housing body 10. However, in FIG. 4, a portion of the outer peripheral edge of the second light-transmitting part 30 is directly connected to the housing body 10, and the ratio of the absolute value of the difference between the thermal expansion coefficients of the material of the second light-transmitting part 30 and the material of the housing body 10 to the thermal expansion coefficient of the material of the housing body 10 is less than or equal to 20% (e.g., the ratio is 20%, 17%, 15%, 12%, 10%, 8%, 5%, 3%, 1%, 0.5%, 0, etc.). It can be understood that by setting the second light-transmitting part 30 to be directly connected to the housing body 10, since the difference between the thermal expansion coefficients of the materials of the second light-transmitting part 30 and the housing body 10 is very small, the part where the housing body 10 and the second light-transmitting part 30 are connected is not prone to cracking, thereby enhancing the mechanical strength of the housing 110.

Referring to FIG. 5, FIG. 5 is a fifth structural schematic view of a housing according to some embodiments of the present disclosure. The light-shielding baffle 40 may include a fifth baffle 45, the fifth baffle 45 being arranged around the second light-transmitting part 30, and the fifth baffle 45 defines one or more fifth notches 451, where the second light-transmitting part 30 is directly connected to the housing body 10 at the one or more fifth notches 451; all regions of the outer peripheral edge of the first light-transmitting part 20 are connected to the housing body 10. It is understood that when all regions of the outer peripheral edge of the first light-transmitting part 20 are connected to the housing body 10, no light-shielding baffle 40 is provided on the outer periphery of the first light-transmitting part 20.

It is understood that when the second light-transmitting part 30 is directly connected to the housing body 10 at the fifth notch 451, all regions of the outer peripheral edge of the first light-transmitting part 20 are connected to the housing body 10, and the first light-transmitting part 20, the second light-transmitting part 30 and the housing body 10 are extruded from the same material (i.e., the thermal expansion coefficients of the materials are the same), the mechanical strength of the housing 110 can be significantly improved, and the housing 110 is less prone to cracking.

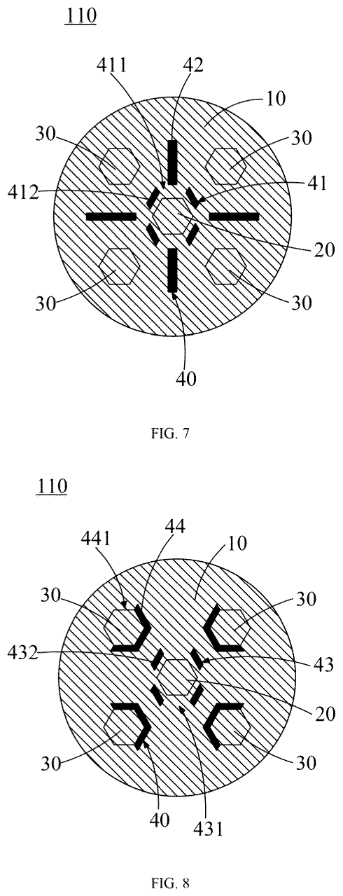

Referring to FIG. 6, FIG. 6 is a sixth structural schematic view of a housing according to some embodiments of the present disclosure. FIG. 6 differs from FIG. 5 in that in FIG. 5, a fifth notch 451 is defined on the fifth baffle 45, and the second light-transmitting part 30 is directly connected to the housing body 10 at the fifth notch 451, while in FIG. 6, the fifth baffle 45 is in a closed state. That is, the fifth notch 451 is not provided on the fifth baffle 45, and the second light-transmitting part 30 is not connected to the housing body 10. However, in FIG. 6, all regions of the outer peripheral edge of the first light-transmitting part 20 are directly connected to the housing body 10, and the ratio of the absolute value of the difference between the thermal expansion coefficients of the material of the first light-transmitting part 20 and the material of the housing body 10 to the thermal expansion coefficient of the material of the housing body 10 is less than or equal to 20% (e.g., the ratio is 20%, 17%, 15%, 12%, 10%, 8%, 5%, 3%, 1%, 0.5%, 0, etc.). It can be understood that by setting the first light-transmitting part 20 to be connected to the housing body 10, since the difference between the thermal expansion coefficients of the materials of the first light-transmitting part 20 and the housing body 10 is very small, the part where the housing body 10 is connected to the first light-transmitting part 20 is not prone to cracking, thereby enhancing the mechanical strength of the housing 110.

Referring to FIG. 7, FIG. 7 is a seventh structural schematic view of a housing according to some embodiments of the present disclosure. It can be seen that the difference between FIG. 7 and FIG. 1 is that in FIG. 1, the first baffle 41 is connected to the first light-transmitting part 20, while in FIG. 7, the first baffle 41 is spaced apart from the first light-transmitting part 20. It can be understood that when the first baffle 41 is spaced from the first light-transmitting part 20, a portion of the first light-transmitting part 20 facing the first baffle 41 is directly connected to the housing body 10, that is, all portions of the outer peripheral edge of the first light-transmitting part 20 are connected to the housing body 10, thereby further enhancing the mechanical strength of the housing 110.

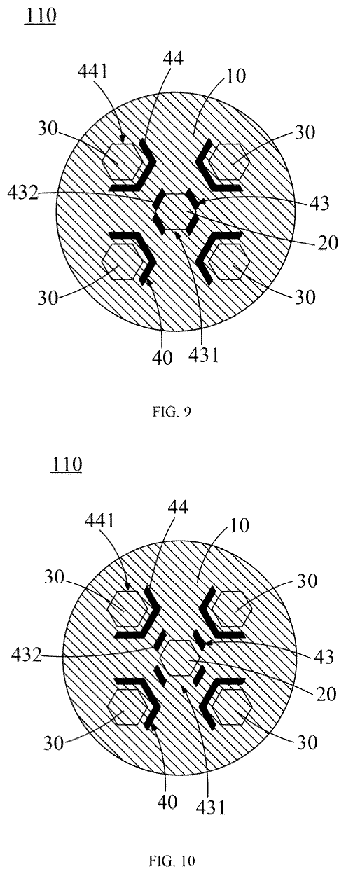

Referring to FIG. 8, FIG. 8 is an eighth structural schematic view of a housing according to some embodiments of the present disclosure. It can be seen that the difference between FIG. 8 and FIG. 3 is that in FIG. 3, the third baffle 43 is connected to the first light-transmitting part 20, while in FIG. 8, the third baffle 43 is arranged spaced from the first light-transmitting part 20. It can be understood that when the third baffle 43 is spaced apart from the first light-transmitting part 20, a portion of the first light-transmitting part 20 facing the third baffle 43 is directly connected to the housing body 10, that is, all portions on the outer peripheral edge of the first light-transmitting part 20 are connected to the housing body 10, thereby further enhancing the mechanical strength of the housing 110.

Referring to FIG. 9, FIG. 9 is a ninth structural schematic view of a housing according to some embodiments of the present disclosure. It can be seen that the difference between FIG. 9 and FIG. 3 is that in FIG. 3, the fourth baffle 44 is connected to the second light-transmitting part 30, while in FIG. 9, the fourth baffle 44 is arranged spaced from the second light-transmitting part 30. It can be understood that when the fourth baffle 44 is spaced apart from the second light-transmitting part 30, a portion of the second light-transmitting part 30 facing the fourth baffle 44 is directly connected to the housing body 10, that is, all portions of the outer peripheral edge of the second light-transmitting part 30 are connected to the housing body 10, thereby further enhancing the mechanical strength of the housing 110.

Referring to FIG. 10, FIG. 10 is a tenth structural schematic view of a housing according to some embodiments of the present disclosure. It can be seen that the difference between FIG. 10 and FIG. 3 is that in FIG. 3, the third baffle 43 is connected to the first light-transmitting part 20, and the fourth baffle 44 is connected to the second light-transmitting part 30, while in FIG. 10, the third baffle 43 is arranged spaced apart from the first light-transmitting part 20. It can be understood that when the third baffle 43 is arranged spaced apart from the first light-transmitting part 20, a portion of the first light-transmitting part 20 facing the third baffle 43 is directly connected to the housing body 10, and when the fourth baffle 44 is arranged spaced apart from the second light-transmitting part 30, a portion of the second light-transmitting part 30 facing the fourth baffle 44 is directly connected to the housing body 10. In other words, all portions of the outer periphery edge of the first light-transmitting part 20 are connected to the housing body 10, and all portions of the outer periphery edge of the second light-transmitting part 30 are connected to the housing body 10, thereby further enhancing the mechanical strength of the housing 110.

Referring to FIG. 11, FIG. 11 is an eleventh structural schematic view of a housing according to some embodiments of the present disclosure. It can be seen that the difference between FIG. 11 and FIG. 5 is that in FIG. 5, the fifth baffle 45 is connected to the second light-transmitting part 30, while in FIG. 11, the fifth baffle 45 is arranged spaced from the second light-transmitting part 30. It can be understood that that when the fifth baffle 45 is spaced from the second light-transmitting part 30, a portion of the second light-transmitting part 30 facing the fifth baffle 45 is directly connected to the housing body 10, that is, all portions of the outer peripheral edge of the second light-transmitting part 30 are connected to the housing body 10, thereby further enhancing the mechanical strength of the housing 110.

Schematically, the absolute value of the difference between the thermal expansion coefficient of any one of the material 71 of the housing body 10, the material 72 of the first light-transmitting part 20, and the material 73 of the second light-transmitting part, and the thermal expansion coefficient of the material 74 of the light-shielding baffle 40 is a second difference, and the ratio of the second difference to the thermal expansion coefficient of the material 74 of the light-shielding baffle 40 is less than or equal to 20% (e.g., the ratio is 20%, 17%, 15%, 12%, 10%, 8%, 5%, 3%, 1%, 0.5%, 0, etc.), and/or, the ratio of the second difference value to the thermal expansion coefficient of any one of the material 71 of the housing body 10, the material 72 of the first light-transmitting part 20, and the material 73 of the second light-transmitting part 30 is less than or equal to 20% (e.g., a ratio of 20%, 17%, 15%, 12%, 10%, 8%, 5%, 3%, 1%, 0.5%, 0, etc.).

It is understood that by setting the ratio of the second difference to the thermal expansion coefficient of the material 74 of the light-shielding baffle 40 to be less than or equal to 20%, and/or setting the ratio of the second difference to the thermal expansion coefficient of any one of the material 71 of the housing body 10, the material 72 of the first light-transmitting part 20, and the material 73 of the second light-transmitting part 30 to be less than or equal to 20%, after the material 71 of the housing body 10, the material 73 of the second light-transmitting part 30, the material 72 of the first light-transmitting part 20, and the material 74 of the light-shielding baffle 40 are heated, softened, and pressed together, they are less likely to crack.

Schematically, the housing body 10, the first light-transmitting part 20, the second light-transmitting part 30, and the light-shielding baffle 40 are all extruded from rod-shaped materials.

Schematically, the overall structure of the housing 110 may be flat, curved, bent, or any other shape, and the cross-section of the housing 110 may be circular, elliptical, sector, square, rectangular, equilateral triangular, regular hexagonal, regular pentagonal, irregular, or any other shape.

Referring to FIGS. 1 to 11, the housing 110 provided in the embodiments of the present disclosure can block the light in the first light-transmitting part 20 from entering the second light-transmitting part 30 by arranging a light-shielding baffle 40 between the first light-transmitting part 20 and the second light-transmitting part 30, thereby preventing the light in the first light-transmitting part 20 from interfering with the light in the second light-transmitting part 30. When the housing 110 is applied to an electronic device, the first light-transmitting part 20 is arranged opposite a light emitter and the second light-transmitting part 30 is arranged opposite a light detector, the emitted light signal in the first light-transmitting part 20 can be prevented from interfering with the received light signal in the second light-transmitting part 30, thereby improving the accuracy of the optical signal detected by the light detector, and thus improving the accuracy of the detection results of the electronic device. In addition, in the embodiments of the present disclosure, the first light-transmitting part 20 and/or the second light-transmitting part 30 are arranged to be directly connected to the housing body 10 (i.e., the first light-transmitting part 20 and/or the second light-transmitting part 30 and the housing body 10 form a one-piece structure), and the thermal expansion coefficient of the material of the first light-transmitting part 20 and/or the second light-transmitting part 30 directly connected to the housing body 10 is set to be close to the thermal expansion coefficient of the material of the housing body 10, so as to significantly improve the mechanical strength of the housing 110, such that the housing 110 has better impact resistance, thereby improving the service life of the electronic device including the housing 110.

Referring to FIGS. 1 to 11, it can be seen that in the housing 110 provided in the embodiments of the present disclosure, the occupied area of the light-shielding baffle 40 is small. Since the material of the light-shielding baffle 40 is usually black glass, and the materials of the housing body 10, the first light-transmitting part 20, and the second light-transmitting part 30 are usually transparent glass, combined with the fact that the cost of black glass is much higher than that of transparent glass, the embodiments of the present disclosure can reduce the amount of black glass used by reducing the occupied area of the light-shielding baffle 40, thereby reducing the production cost of the housing 110.

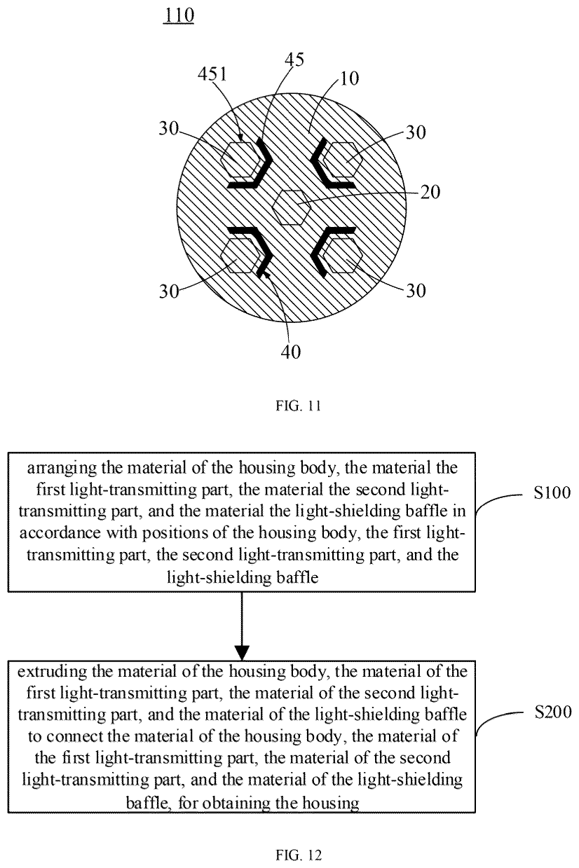

Referring to FIG. 12, FIG. 12 is a flowchart of a method for manufacturing a housing according to some embodiments of the present disclosure. The embodiments of the present disclosure further provide a method for manufacturing the housing 110 of any of the above embodiments, the method may include operations at blocks illustrated herein.

At block S100: arranging the material of the housing body, the material the first light-transmitting part, the material the second light-transmitting part, and the material the light-shielding baffle in accordance with positions of the housing body, the first light-transmitting part, the second light-transmitting part, and the light-shielding baffle.

Referring to FIG. 13 and in combination with FIG. 1, the material 71 of the housing body 10, the material 72 of the first light-transmitting part 20, the material 73 of the second light-transmitting part 30, and the material 74 of the light-shielding baffle 40 are arranged in an order according to the positions of the housing body 10, the first light-transmitting part 20, the second light-transmitting part 30, and the light-shielding baffle 40 in FIG. 1, and then the material 71 of the housing body 10, the material 72 of the first light-transmitting part 20, the material 73 of the second light-transmitting part 30, and the material 74 of the light-shielding baffle 40 are extruded to obtain the housing 110 as shown in FIG. 1.

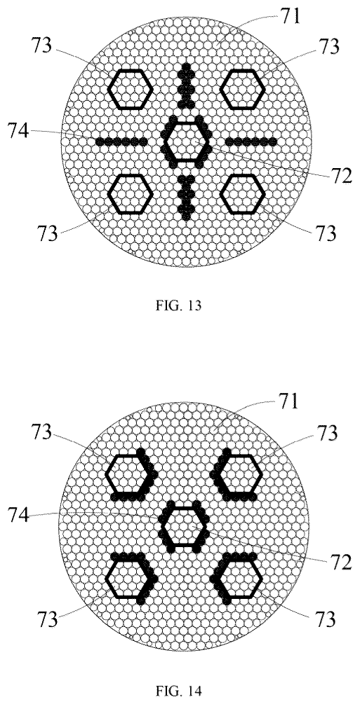

Referring to FIG. 14 and in combination with FIG. 3, the material 71 of the housing body 10, the material 72 of the first light-transmitting part 20, the material 73 of the second light-transmitting part 30, and the material 74 of the light-shielding baffle 40 are arranged in an order according to the positions of the housing body 10, the first light-transmitting part 20, the second light-transmitting part 30, and the light-shielding baffle 40 in FIG. 3, and then the material 71 of the housing body 10, the material 72 of the first light-transmitting part 20, the material 73 of the second light-transmitting part 30, and the material 74 of the light-shielding baffle 40 are extruded to obtain the housing 110 as shown in FIG. 1.

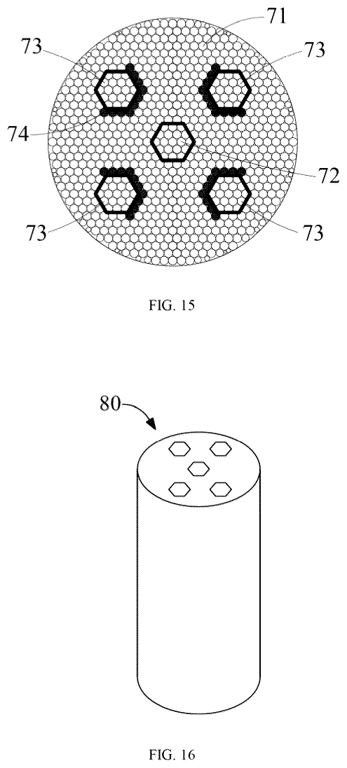

Referring to FIG. 15, and in combination with FIG. 5, the material 71 of the housing body 10, the material 72 of the first light-transmitting part 20, the material 73 of the second light-transmitting part 30, and the material 74 of the light-shielding baffle 40 are arranged in an order according to the positions of the housing body 10, the first light-transmitting part 20, the second light-transmitting part 30, and the light-shielding baffle 40 in FIG. 5, and then the material 71 of the housing body 10, the material 72 of the first light-transmitting part 20, the material 73 of the second light-transmitting part 30, and the material 74 of the light-shielding baffle 40 are extruded to obtain the housing 110 as shown in FIG. 1.

It should be noted that in FIGS. 13 to 15, the black bold lines around the periphery of the material 72 of the first light-transmitting part 2020 are only intended to indicate the range of the setting region of the material 72, and the black bold lines around the periphery of the material 73 of the second light-transmitting part 3030 are only intended to indicate the range of the setting region of the material 73.

It can be understood that when the material 71 of the housing body 10, the material 72 of the first light-transmitting part 20, the material 73 of the second light-transmitting part 30, and the material 74 of the light-shielding baffle 40 are in a non-softened state at room temperature, the material 71 of the housing body 10, the material 72 of the first light-transmitting part 20, the material 73 of the second light-transmitting part 30, and the material 74 of the light-shielding baffle 40 are required to be softened before they are extruded together.

For example, when the material 71 of the housing body 10, the material 72 of the first light-transmitting part 20, the material 73 of the second light-transmitting part 30, and the material 74 of the light-shielding baffle 40 are materials such as glass or resin that can be softened by heating, the material 71 of the housing body 10, the material 72 of the first light-transmitting part 20, the material 73 of the second light-transmitting part 30, and the material 74 of the light-shielding baffle 40 can be softened by heating.

For example, the absolute value of the difference between the softening point temperature of any one of the material 71 of the housing body 10, the material 72 of the first light-transmitting part 20, and the material 73 of the second light-transmitting part 30, and the softening point temperature of the material 74 of the light-shielding baffle 40 is less than or equal to 5° C. (e.g., the absolute value of the difference is 5° C. 4° C., 3° C., 2° C., 1° C., 0.5° C., 0, etc.), such that a situation during heating may be prevented, where while one material has already reached the softened state, the other material is already melted or is still in the hardened state.

For example, the material 71 of the housing body 10, the material 72 of the first light-transmitting part 20, the material 73 of the second light-transmitting part 30, and the material 74 of the light-shielding baffle 40 are all rod-shaped materials.

It is understood that the rod-shaped material refers to a material that has a length in the direction of extension that is greater than the width in any direction in the cross-section.

In the embodiments of the present disclosure, the diameter of the rod-shaped material may be 0.01 mm to 50 mm, for example 0.01 mm, 0.03 mm, 0.05 mm, 0.07 mm, 0.1 mm, 0.3 mm, 0.5 mm, 0.7 mm, 1 mm, 3 mm, 5 mm, 7 mm, 10 mm, 15 mm, 20 mm, 25 mm, 30 mm, 35 mm, 40 mm, 45 mm, 50 mm, etc.

For example, the material 71 of the housing body 10, the material 72 of the first light-transmitting part 20, the material 73 of the second light-transmitting part 30, and the material 74 of the light-shielding baffle 40 may have the same or different diameters, i.e., each material may include rod-shaped materials of various diameters, such that rod-shaped materials of different diameters may be combined, and small rod-shaped materials may be inserted between the larger rod-shaped materials, so as to minimize the gaps between the rod-shaped materials, thereby improving the compactness of the rod-shaped materials after extrusion, and preventing the resulting housing 110 from collapsing. For example, the material 71 of the housing body 10, the material 72 of the first light-transmitting part 20, the material 73 of the second light-transmitting part 30, and the material 74 of the light-shielding baffle 40 may each include a rod-shaped material with a diameter of 3 mm and a rod-shaped material with a diameter of 0.46 mm. The rod-shaped material with a diameter of 0.46 mm may be filled in the gaps between the rod-shaped material with a diameter of 3 mm to improve the compactness of the rod-shaped materials after extrusion.

At block S200: extruding the material of the housing body, the material of the first light-transmitting part, the material of the second light-transmitting part, and the material of the light-shielding baffle to connect the material of the housing body, the material of the first light-transmitting part, the material of the second light-transmitting part, and the material of the light-shielding baffle, for obtaining the housing.

Referring to FIG. 16, FIG. 16 is a structural schematic view of a composite rod body according to some embodiments of the present disclosure. The material 71 of the housing body 10, the material 72 of the first light-transmitting part 20, the material 73 of the second light-transmitting part 30, and the material 74 of the light-shielding baffle 40 are extruded, such that the material 71 of the housing body 10, the material 72 of the first light-transmitting part 20, the material 73 of the second light-transmitting part 30, and the material 74 of the light-shielding baffle 40 are joined together, and thus a composite rod body 80 is obtained. The composite rod body 80 can be processed to obtain the housing 110. Exemplarily, the composite rod body 80 can be processed by cutting or milling to obtain the housing 110.

After obtaining the composite rod body 80, the composite rod body 80 can be drawn to reduce the cross-sectional dimension of the composite rod body 80 to a dimension that matches the product design. After drawing, the composite rod body 80 can be processed to obtain the housing 110. After drawing, the diameter of the composite rod body 80 can be reduced to 1.2 mm, 1 mm, etc.

The “arranging the material of the housing body: the material the first light-transmitting part, the material the second light-transmitting part, and the material the light-shielding baffle in accordance with positions of the housing body, the first light-transmitting part, the second light-transmitting part, and the light-shielding baffle”, and the “extruding the material of the housing body, the material of the first light-transmitting part, the material of the second light-transmitting part, and the material of the light-shielding baffle” may be carried out in one or more steps, i.e. the composite rod body 80 can be formed by one or more rod placements. For example, in some embodiments, the composite rod body 80 can be formed by one rod placement, i.e. all rods of the housing body 10, all rods of the first light-transmitting part 20, all the rods of the second light-transmitting part 30, and all the rods of the light-shielding baffle 40 are arranged together (one-time rod placement), and the composite rod body 80 is formed by one extrusion. In other embodiments, the composite rod body 80 can be formed by two-time rod placement, i.e., the material 71 of the housing body 10, the material 72 of the first light-transmitting part 20, the material 73 of the second light-transmitting part 30, and the material 74 of the light-shielding baffle 40 are each divided into multiple parts, and each parts of rods are arranged together (first rod placement) and then extruded to form a first rod body (first extrusion); multiple first rod bodies are arranged (second rod placement) and then are extruded to form the composite rod body 80 (second extrusion). Or, according to actual production requirements, three-time rod placements, four-time rod placements, etc. can be performed. It can be understood that multiple rod arrangements can reduce air gaps between the rods and improve the tightness of the rods after extrusion.

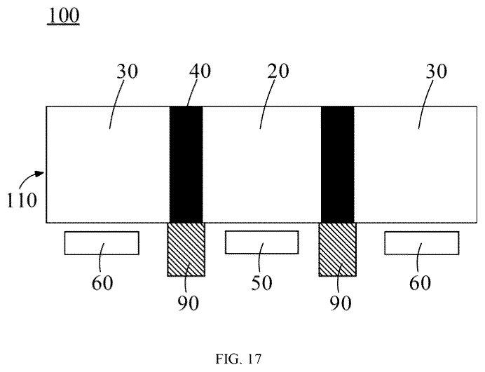

Referring to FIG. 17 in combination with FIGS. 1 to 5, FIG. 17 is a partially structural schematic view of an electronic device according to some embodiments of the present disclosure. The embodiments of the present disclosure further provide an electronic device 100, including a housing 110, a light emitter 50, and a light detector 60; where the housing 110 can be the housing 110 of any of the above embodiments; the light emitter 50 is arranged on a side of the housing 110, and light emitted by the light emitter 50 is configured to enter the first light-transmitting part 20 and be transmitted to a side of the housing 110 away from the light emitter 50 via the first light-transmitting part 20; the light detector 60 and the light emitter 50 are arranged on a same side of the housing 110, and light incident from the side of the housing 110 away from the light detector 60 is configured to enter the second light-transmitting part 30, be transmitted through the second light-transmitting part 30, and enter the light detector 60.

Schematically, the electronic device 100 may be a smart wearable device, such as a watch, bracelet, ring, arm sleeve, clothing, etc.; the electronic device 100 may have a health monitoring function, and the health monitoring function may include, but is not limited to, a blood oxygen monitoring function and a heart rate monitoring function; the health monitoring function of the electronic device 100 may be implemented by Photoplethysmography (PPG).

Schematically, the housing 110 may serve as a back cover (i.e., the cover on a side close to the skin of the user) of the smart wearable device (e.g., a smartwatch).

Schematically, the light emitter 50 may include an LED lamp, and the light detector 60 may include a light sensor.

Referring to FIG. 17, the working principle of the electronic device 100 is as follows: the light emitted by the light emitter 50 passes through the first light-transmitting part 20 of the housing 110 and enters the skin. Some of the light is absorbed by the skin, while some of the light is reflected off the skin surface. The reflected light enters the light detector 60 after passing through the second light-transmitting part 30 of the housing 110. By converting the optical signal received by the light detector 60, relevant physiological information about the human body can be obtained, such as data on heart rate and blood oxygen saturation.

The electronic device 100 provided in the embodiments of the present disclosure may avoid the light emitted by the light emitter 50 from slipping into the second light-transmitting part 30 from the first light-transmitting part 20 and being received by the light detector 60, by virtue of the aforementioned housing 110, thereby improving the accuracy of the detection results.

Referring to FIG. 17, the electronic device 100 may further include a shielding member 90. The shielding member 90 is arranged on the same side of the housing 110 as the light emitter 50 and the light detector 60. The shielding member 90 is arranged between the light emitter 50 and the light detector 60 to prevent crosstalk caused by the light emitted by the light emitter 50 entering the light detector 60 directly. For example, a top surface of the shielding member 90 may abut against a bottom surface of the housing 110 to maximize light shielding. The shielding member 90 may be made of glass, ceramic, plastic, etc.

The above provides a detailed description of a housing, a method for manufacturing a housing, and an electronic device provided in the embodiments of the present disclosure. Specific examples are provided in this document to illustrate the principles and implementations of the present disclosure. The above description of the embodiments is only intended to facilitate understanding of the present disclosure. For persons skilled in the art, there will be changes in specific embodiments and scope of application based on the ideas of the present disclosure. In summary, the content of the present specification should not be understood as a limitation of the present disclosure.

1. A housing, comprising:
a housing body;
a first light-transmitting part;
a second light-transmitting part, spaced apart from the first light-transmitting part; and
a light-shielding baffle, arranged between the first light-transmitting part and the second light-transmitting part;
wherein at least one of the following is satisfied:
at least a portion of an outer peripheral edge of the first light-transmitting part is directly connected to the housing body; and
at least a portion of the outer peripheral edge of the second light-transmitting part is directly connected to the housing body.

2. The housing according to claim 1, wherein the material of the housing body, the material of the first light-transmitting part, and the material of the second light-transmitting part are the same.

3. The housing according to claim 1, wherein the second light-transmitting part comprises a plurality of second light-transmitting parts, and the plurality of second light-transmitting parts are arranged around the first light-transmitting part, wherein the first light-transmitting part is circular in shape, and each of the plurality of second light-transmitting parts is sector-shaped.

4. The housing according to claim 3, wherein the light-shielding baffle comprises a first baffle and a second baffle;
the first baffle is arranged around the first light-transmitting part, and the first baffle defines one or more first notches;
the second baffle is arranged between each adjacent two of the plurality of second light-transmitting parts.

5. The housing according to claim 4, wherein the second baffle is not connected to the first baffle.

6. The housing according to claim 4, wherein the first baffle is connected to the first light-transmitting part, and the first light-transmitting part is connected to the housing body at the one or more first notches;
all regions of an outer peripheral edge of each of the plurality of second light-transmitting parts are connected to the housing body.

7. The housing according to claim 4, wherein the first baffle is arranged spaced apart from the first light-transmitting part, and all regions of an outer peripheral edge of the first light-transmitting part are connected to the housing body;
all regions of an outer peripheral edge of each of the plurality of second light-transmitting parts are connected to the housing body.

8. The housing according to claim 1, wherein the light-shielding baffle comprises a third baffle and a fourth baffle;
the third baffle is arranged around the first light-transmitting part, and the third baffle defines one or more third notches;
the fourth baffle is arranged around each second light-transmitting part, and the fourth baffle defines one or more fourth notches.

9. The housing according to claim 8, wherein
the third baffle is connected to the first light-transmitting part, and the first light-transmitting part is connected to the housing body at the one or more third notches; the fourth baffle is connected to the second light-transmitting part, and the second light-transmitting part is connected to the housing body at the one or more fourth notches; or
the third baffle is connected to the first light-transmitting part, and the first light-transmitting part is connected to the housing body at the one or more third notches; the fourth baffle is arranged spaced apart from the second light-transmitting part, and all regions of an outer peripheral edge of the second light-transmitting part are connected to the housing body; or
the third baffle is arranged spaced apart from the first light-transmitting part and all regions of an outer peripheral edge of the first light-transmitting part are connected to the housing body: the fourth baffle is connected to the second light-transmitting part, and the second light-transmitting part is connected to the housing body at the one or more fourth notches; or
the third baffle is arranged spaced from the first light-transmitting part, and all regions of an outer peripheral edge of the first light-transmitting part are connected to the housing body: the fourth baffle is arranged spaced from the second light-transmitting part and all regions of an outer peripheral edge of the second light-transmitting part are connected to the housing body.

10-12. (canceled)

13. The housing according to claim 1, wherein the light-shielding baffle comprises a fifth baffle, the fifth baffle being arranged around the second light-transmitting part, and the fifth baffle defines one or more fifth notches;
all regions of an outer peripheral edge of the first light-transmitting part are connected to the housing body.

14. The housing according to claim 13, wherein
the fifth baffle is connected to the second light-transmitting part, and the second light-transmitting part is directly connected to the housing body at the one or more fifth notches; or
the fifth baffle is spaced apart from the second light-transmitting part, and all regions of an outer peripheral edge of the second light-transmitting part are connected to the housing body.

15. (canceled)

16. The housing according to claim 1, wherein an absolute value of a difference between the thermal expansion coefficient of any one of the material of the housing body, the material of the first light-transmitting part, and the material of the second light-transmitting part, and the thermal expansion coefficient of the material of the light-shielding baffle is a third difference, and at least one of the following is satisfied:
a ratio of the third difference to the thermal expansion coefficient of the material of the light-shielding baffle is less than or equal to 20%; and
the ratio of the second difference value to the thermal expansion coefficient of any one of the material of the housing body, the material of the first light-transmitting part, and the material of the second light-transmitting part is less than or equal to 20%.

17. A method for manufacturing the housing according to claim 1, comprising:
arranging the material of the housing body, the material the first light-transmitting part, the material the second light-transmitting part, and the material the light-shielding baffle in accordance with positions of the housing body, the first light-transmitting part, the second light-transmitting part, and the light-shielding baffle; and
extruding the material of the housing body, the material of the first light-transmitting part, the material of the second light-transmitting part, and the material of the light-shielding baffle to connect the material of the housing body, the material of the first light-transmitting part, the material of the second light-transmitting part, and the material of the light-shielding baffle, for obtaining the housing.

18-19. (canceled)

20. An electronic device, comprising:
a housing, comprising:
a housing body;
a first light-transmitting part;
a second light-transmitting part, spaced apart from the first light-transmitting part; and
a light-shielding baffle, arranged between the first light-transmitting part and the second light-transmitting part; wherein at least one of the following is satisfied: at least a portion of an outer peripheral edge of the first light-transmitting part is directly connected to the housing body; and, at least a portion of the outer peripheral edge of the second light-transmitting part is directly connected to the housing body;
a light emitter, arranged on a side of the housing; wherein light emitted by the light emitter is configured to enter the first light-transmitting part and be transmitted to another side of the housing away from the light emitter via the first light-transmitting part; and
a light detector; wherein the light detector and the light emitter are arranged on a same side of the housing, and the light incident from the side of the housing away from the light detector is configured to enter the second light-transmitting part, be transmitted through the second light-transmitting part, and then enter the light detector.

21. The housing according to claim 1, wherein at least one of the following is satisfied:
an absolute value of a difference between a thermal expansion coefficient of a material of the first light-transmitting part and a thermal expansion coefficient of a material of the housing body is a first difference, and a ratio of the first difference to the thermal expansion coefficient of the material of the housing body is less than or equal to 20%; and
an absolute value of a difference between a thermal expansion coefficient of a material of the second light-transmitting part and a thermal expansion coefficient of a material of the housing body is a second difference, and a ratio of the second difference to the thermal expansion coefficient of the material of the housing body is less than or equal to 20%.

22. The housing according to claim 21, wherein at least one of the following is satisfied:
the ratio of the first difference to the thermal expansion coefficient of the material of the housing body is equal to 0; and
the ratio of the second difference to the thermal expansion coefficient of the material of the housing body is equal to 0.

23. The housing according to claim 1, wherein a material of the housing body, a material of the first light-transmitting part, a material of the second light-transmitting part, and a material of the light-shielding baffle are each glass or resin.

24. The housing according to claim 1, wherein at least one of the following is satisfied:
the first light-transmitting part is directly connected to the housing body; and
the second light-transmitting part is directly connected to the housing body.

25. The housing according to claim 1, wherein the housing body, the first light-transmitting part, the second light-transmitting part, and the light-shielding baffle are in a one-piece structure.

26. The housing according to claim 1, wherein the light-shielding baffle is in a closed state, the first light-transmitting part is not connected to the housing body.
